(19) United States
(12) Patent Application Publication
(10) Pub. No.: US 2004/0006142 A1
(43) Pub. Date: Jan. 8, 2004

(54) Ocular tension depressor

(76) Inventor: Ryuji Ueno, Montgomery, MD (US)

Correspondence Address: SUGHRUE MION, PLLC, 2100 PENNSYLVANIA AVENUE, N.W., WASHINGTON, DC, 20037, US

(21) Appl. No.: 10/275,303

(22) PCT Filed: Apr. 26, 2001
(86) PCT No.: PCT/JP01/03617

(51) Int. Cl. A61K 31/165; C07C 237/36

(52) U.S. Cl. 514/616; 564/153; 564/155

(57) ABSTRACT

This invention relates to an ocular tension depressor containing a specific benzamide derivative as an active ingredient.

### TECHNICAL FIELD

This invention relates to an ocular tension depressor which contains a specific benzamide derivative as an active ingredient.

### BACKGROUND ART

Glaucoma is a disease in which nervus opticus is damaged due to sthenia of an intraocular pressure of a human being or animal beyond a certain limit. Glaucoma may likely to result in blindness or extreme deterioration of optesthesia when treatment is not conducted or an inappropriate treatment is conducted. A symptom which shows a high ocular tension but does not show any other anomaly than the high ocular tension is referred to as “high ocular tension disease”, which is differentiated from glaucoma. The high ocular tension disease has a possibility of being progressed into glaucoma after a lapse of a long time. Therefore, the high ocular tension disease can be regarded as a disease at a most initial stage of glaucoma. On the other hand, in the case where a change which presumably results from glaucoma is observed with respect to the visual field or optic disk of a patient despite the fact that ocular tension stays in a normal range, such a symptom is referred to as “glaucoma with normal ocular tension”. This disease is also one of glaucoma according to broad definition of glaucoma. In any of the diseases, it is essential to depress ocular tension to at least a level of a certain range which the eye suffering from the disease can endure. Therefore, a development of medicament capable of effectively depressing the ocular tension has been demanded.

### DISCLOSURE OF INVENTION

The inventor of this invention has made a research and development to provide a medicament which is pharmaceutically effective in treating glaucoma and high ocular tension disease. As a result of research and development, the inventor has found out that using a specific benzamide derivative represented by the following formula (I) results in sharp depressing of ocular tension, and made this invention. It should be noted that the benzamide derivative itself is conventional and disclosed in U.S. Pat. No. 5,521,170, EP-A-832061, and WO-A-96/33723. However, none of the publications disclose that the benzamide derivative exhibits an excellent performance of depressing ocular tension, and it is the first finding of the inventor of this invention that the benzamide derivative is effective in depressing ocular tension.

This invention relates to an ocular tension depressor containing, as an active ingredient, a compound represented by the general formula (I):

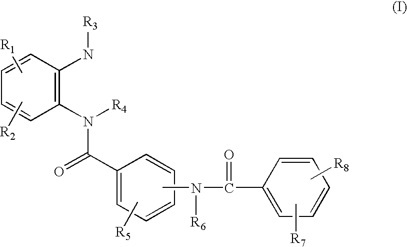

wherein

R₁ is hydrogen or lower alkyl,

R₂ is hydrogen, lower alkyl, halo(lower)alkyl, halogen or lower alkoxy,

R₃ is lower alkyl which may be substituted with acyl or acylamino,

A is O,

R₄ is hydrogen; lower alkyl which may be substituted with hydroxy, aryl or acyl; or cyclo(lower)alkyl, or

A is

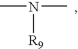

and R₉ and R₄ may be linked together to form lower alkylene which may be substituted with oxo,

R₅ is hydrogen, halogen, nitro, hydroxy, protected hydroxy, lower alkyl, or lower alkoxy which may be substituted with lower alkylamino,

R₆ is hydrogen, lower alkyl or acyl,

R₇ is hydrogen, halogen, hydroxy or lower alkoxy,

R₈ is hydroxy, aryl, acyl, amino, lower alkoxy which may be substituted with lower alkylamino or acylamino; or aryl which may be substituted with at least one substituent selected from the group consisting of lower alkyl, lower alkoxy, halogen, halo(lower)alkyl, hydroxy, amino(lower)alkyl, azido(lower)alkyl, lower alkylamino(lower)alkyl, acylamino(lower)alkyl, hydroxy(lower)alkyl, cyano and acyl, or a pharmaceutically acceptable salt thereof.

According to another aspect of this invention, this invention relates to a method for treating high ocular tension and/or glaucoma by administering an effective amount of the benzamide derivative.

According to yet another aspect of this invention, this invention relates to a use of the benzamide derivative to treat high ocular tension and/or glaucoma.

Hereinafter, terms and definitions thereof used in this specification are explained.

The term “lower” is intended to mean 1 to 6 carbon atom(s), unless otherwise indicated.

Suitable “lower alkyl”, and “lower alkyl moiety” in the terms “halo(lower)alkyl”, “lower alkylamino”, “amino(lower)alkyl”, “azido(lower)alkyl”, “lower alkylamino(lower)alkyl”, “acylamino(lower)alkyl”, “hydroxy(lower)alkyl” and “lower alkyl carbamoyl” which is described later may include straight or branched (C₁-C₆)alkyl such as methyl, ethyl, propyl, isopropyl, butyl, isobutyl, tert-butyl, pentyl, ethylpropyl, hexyl or the like. The more preferred one may be methyl, propyl and pentyl.

Suitable “lower alkoxy” may include straight or branched (C₁-C₆)alkoxy such as methoxy, ethoxy, propoxy, isopropoxy, methylpropoxy, butoxy, isobutoxy, tert-butoxy, pentyloxy, hexyloxy or the like. The more preferred one may be methoxy and propoxy.

Suitable “halogen”, and “halo moiety” in the term “halo(lower)alkyl” may include fluorine, chlorine, bromine and iodine.

Suitable “cyclo(lower)alkyl” may include cyclo(C₃-C₆)alkyl such as cyclopropyl, cyclobutyl, cyclopentyl, cyclohexyl or the like.

Suitable “lower alkylene” may include straight or branched (C₁-C₆)alkylene such as methylene, ethylene, trimethylene, propylene, tetramethylene, pentamethylene, hexamethylene or the like. The more preferred one may be trimethylene.

Suitable “aryl” may include phenyl, naphthyl, phenyl which is substituted with lower alkyl (such as tolyl, xylyl, mesityl, cumenyl, di-tert-butylphenyl) or the like. The more preferred one may be phenyl or tolyl.

Suitable “lower alkylamino”, and “lower alkylamino moiety” in the term “lower alkylamino(lower)alkyl” may include mono- or di-lower alkylamino such as methylamino, ethylamino, propylamino, isopropylamino, butylamino, tert-butylamino, isobutylamino, pentylamino, hexylamino, dimethylamino, diethylamino, dipropylamino, dibutylamino, diisopropylamino, dipentylamino, dihexylamino, N-methylethylamino or the like.

Suitable “acyl”, and “acyl moiety” in the term “acylamino” and “acylamino(lower)alkyl” may include carboxy, esterified carboxy, carbamoyl, lower alkylcarbamoyl, lower alkanoyl, aroyl, heterocyclic carbonyl or the like.

Esterified carboxy may include substituted or non-substituted lower alkoxycarbonyl (e.g., methoxycarbonyl, ethoxycarbonyl, propoxycarbonyl, butoxycarbonyl, tert-butoxycarbonyl, hexyloxycarbonyl, 2-iodoethoxycarbonyl, 2,2,2-trichloroethoxycarbonyl, dimethylaminopropoxycarbonyl, dimethylaminoethoxycarbonyl, etc.), substituted or non-substituted aryloxycarbonyl (e.g., phenoxycarbonyl, 4-nitrophenoxycarbonyl, 2-naphthyloxycarbonyl. etc.), substituted or non-substituted ar(lower)alkoxycarbonyl (e.g., benzyloxycarbonyl, phenethyloxycarbonyl, benzhydryloxycarbonyl, 4-nitrobenzyloxycarbonyl, 3-methoxy-4-nitrobenzyloxycarbonyl), N-containing heterocyclic oxycarbonyl (e.g., N-lower alkylpyperidyloxycarbonyl, etc.).

Lower alkylcarbamoyl may include mono- or di-lower alkylcarbamoyl such as methylcarbamoyl, ethylcarbamoyl, propylcarbamoyl, dimethylcarbamoyl, diethylcarbamoyl, N-methyl-N-ethylcarbamoyl, or the like.

Lower alkanoyl may include substituted or non-substituted (C₁-C₆)alkanoyl such as formyl, acetyl, propionyl, butyryl, isobutyryl, valeryl, isovaleryl, pivaloyl, hexanoyl, trifluoroacetyl or the like.

Suitable aroyl may include benzoyl, naphthoyl, toluoyl, di-tert-butylbenzoyl or the like.

Suitable “heterocyclic moiety” in the term “heterocyclic carbonyl” may include a heterocyclic group containing at least one hetero-atom selected from the group consisting of a nitrogen atom, a sulfur atom and an oxygen atom. It may include substituted or non-substituted monocyclic or polycyclic heterocyclic group. The more preferable heterocyclic group may include, e.g.,:

unsaturated 3 to 6-membered heteromonocyclic group containing 1 to 4 nitrogen atom(s), for example, pyrrolyl, pyrrolinyl, imidazolyl, pyrazolyl, pyridyl, pyrimidinyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g., 1H-tetrazolyl, 2H-tetrazolyl), etc.,;

saturated 3 to 7-membered heteromonocyclic group containing 1 to 4 nitrogen atom(s), for example, pyrrolidinyl, imidazolidinyl, piperidyl, piperazinyl, homopiperazinyl, etc.,;

unsaturated condensed heterocyclic group containing 1 to 5 nitrogen atom(s), for example, indolyl, isoindolyl, indolizinyl, benzimidazolyl, quinolyl, isoquinolyl, imidazopyridyl, indazolyl, benzotriazolyl, tetrazolopyridazinyl (e.g., tetrazolo[1,5-b]pyridazinyl, etc.,;

unsaturated 3 to 6-membered heteromonocyclic group containing 1 to 2 oxygen atom(s) and 1 to 3 nitrogen atom(s), for example, oxazolyl, isoxazolyl, oxadiazolyl (e.g., 1,2,4-oxadiazolyl, 1,3,4-oxadiazolyl, 1,2,5-oxadiazolyl, etc.), oxazolinyl (e.g., 2-oxazolinyl), etc.,;

saturated 3 to 6-membered heteromonocyclic group containing 1 to 2 oxygen atom(s) and 1 to 3 nitrogen atom(s) (e.g., morpholinyl), etc.,;

unsaturated condensed heterocyclic group containing 1 to 2 oxygen atom(s) and 1 to 3 nitrogen atom(s) (e.g., benzofurazanyl, benzoxazolyl, benzoxadiazolyl, etc.,), etc.,;

unsaturated 3 to 6-membered heteromonocyclic group containing 1 to 2 sulfur atom(s) and 1 to 3 nitrogen atom(s), for example, thiazolyl, thiadiazolyl (e.g., 1,2,4-thiadiazolyl, 1,3,4-thiadiazolyl, 1,2,5-thiadiazolyl, etc.,), etc.,;

saturated 3 to 6-membered heteromonocyclic group containing 1 to 2 sulfur atom(s) and 1 to 3 nitrogen atom(s) (e.g., thiazolidinyl);

N-containing heterocyclic group such as unsaturated condensed heterocyclic group containing 1 to 2 sulfur atom(s) and 1 to 3 nitrogen atom(s) (e.g., benzthiazolyl, benzthiadiazolyl, etc.,);

unsaturated 3 to 6-membered heteromonocyclic group containing 1 oxygen atom, for example, pyranyl, furyl, etc.,;

saturated 3 to 6-membered heteromonocyclic group containing one oxygen atom, for example, 1H-tetrahydropyranyl, tetrahydrofuryl, etc.,; and

unsaturated 3 to 6-membered heteromonocyclic group containing 1 to 2 sulfur atom(s), for example, thienyl, etc.,; and unsaturated condensed heterocyclic group containing 1 to 2 oxygen atom(s) (for example, benzofuranyl, benzodioxolyl, etc.).

The above “heterocyclic group” may be substituted with lower alkyl or oxo. The more preferable heterocyclic group may include N-methylpiperazinyl, tetrazolyl, morpholinyl, pyrrolidinyl, N-methylpiperidyl, N-methylhomopiperazinyl, 1H-tetrahydropyranyl, thienyl, pyridyl, piperidyl, oxopiperidyl, etc.

Suitable examples of “heterocylic carbonyl” may include N-containing heterocyclic carbonyl containing at least 1 nitrogen atom in the heterocyclic group in which the more preferable one may include N-lower alkylpiperazinylcarbonyl (for example, N-methylpiperazinylcarbonyl, etc.), N-lower alkylhomopiperazinyl (for example, N-methylhomopiperazinyl, etc.), piperazinylcarbonyl, pyrrolidinylcarbonyl, piperidinylcarbonyl, morpholinocarbonyl, lower alkylpiperidylcarbonyl (for example, methylpiperidylcarbonyl, etc.), oxopiperidylcarbonyl.

Suitable examples of “protected hydroxy” may be conventional one including substitutable lower alkoxy such as lower alkoxy(lower)alkoxy (for example, methoxymethoxy, etc.), lower alkoxy(lower)alkoxy(lower)alkoxy (for example, methoxyethoxymethoxy, etc.), substituted or non-substituted ar(lower)alkoxy (for example, benzyloxy, nitrobenzyloxy, etc.); and acyloxy such as lower alkanoyloxy (for example, acetoxy, propyonyloxy, pivaloyloxy, etc.), aroyloxy (for example, benzoyloxy, fluorenecarbonyloxy, etc.), lower alkoxycarbonyloxy (for example, methoxycarbonyloxy, ethoxycarbonyloxy, propoxycarbonyloxy, isopropoxycarbonyloxy, butoxycarbonyloxy, isobutoxycarbonyloxy, tert-butoxycarbonyloxy, pentyloxycarbonyloxy, hexyloxycarbonyloxy, etc.), substituted or non-substituted ar(lower)alkoxycarbonyloxy (for example, benzyloxycarbonyloxy, bromobenzyloxycarbonyloxy, etc.) or the like.

Suitable “acyl” in R₃, and suitable “acyl moiety” in the term “acylamino” may include N-containing heterocyclic carbonyl in which the preferred one may be N-lower alkylpiperazinylcarbonyl.

Suitable “lower alkylene which may be substituted with oxo” in which R₉ and R₄ may be linked together may include lower alkylene substituted with oxo. The more preferable one is trimethylene substituted with oxo.

A preferable compound represented by the general formula (I) according to this invention is a compound wherein

R₂ is hydrogen or lower alkyl,

R₃ is lower alkyl which may be substituted with acyl,

A is O,

R₄ is lower alkyl, or

A is

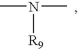

and R₉ and R₄ may be linked together to form lower alkylene which is substituted with oxo,

R₅ is hydrogen or lower alkoxy,

R₆ and R₇ are independently hydrogen,

R₈ is lower alkoxy which is substituted with amino; or phenyl which is substituted with lower alkyl.

More preferably, the object compound according to this invention is a compound in which R₃ is lower alkyl which is substituted with N-lower alkylpiperazinylcarbonyl.

Specifically, the object compound according to this invention may preferably include:

a compound wherein

R₁ is hydrogen,

R₂ is lower alkyl,

R₃ is lower alkyl substituted with N-lower alkylpiperazinyl carbonyl,

A is O,

R₄ is lower alkyl,

R₅ is lower alkoxy,

R₆ is hydrogen,

R₇ is hydrogen,

R₈ is lower alkoxy substituted with amino; or

a compound wherein

R₁ is hydrogen,

R₂ is hydrogen,

R₃ is lower alkyl substituted with N-lower alkyl piperazinyl carbonyl,

A is

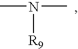

and constitutes lower alkylene substituted with oxo in which R₉ and R₄ are linked together,

R₅ is hydrogen,

R₆ is hydrogen,

R₇ is hydrogen,

R₈ is phenyl substituted with lower alkyl.

More preferably, the object compound (I) includes a compound wherein

R₁ is hydrogen,

R₂ is methyl,

R₃ is pentyl substituted with N-methyl piperazinyl carbonyl,

A is O,

R₄ is methyl,

R₅ is methoxy,

R₆ is hydrogen,

R₇ is hydrogen,

R₈ is propoxy substituted with amino (the chemical name thereof is 4-[2-(3-aminopropyl-1-yl)oxy]benzoylamino-3-methoxy-N-methyl-N-{4-methyl-2-[5-(4-methylpiperazin-1-yl)carbonylpent-1-yloxy]phe nyl}benzamide, and is represented as the compound A which is described below); or

a compound wherein

R₁ is hydrogen,

R₂ is hydrogen,

R₃ is methyl substituted with N-methylpiperazinyl carbonyl,

A is

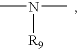

and constitutes trimethylene substituted with oxo in which R₉ and R₄ are linked together,

R₅ is hydrogen,

R₆ is hydrogen,

R₇ is hydrogen,

R₈ is tolyl (the chemical name thereof is 5-{4-[2-(4-methylphenyl)benzoylamino]benzoyl}-1-[(4-methyl-1-pip erazinyl)carbonylmethyl]-1,3,4,5-tetrahydro-1,5-benzodiazepin-2(2H)-one, and is represented by the compound B which is described later).

Suitable pharmaceutically acceptable salts of the object compound (I) are conventional non-toxic salts and include: acid salts such as an inorganic acid salt (e.g., hydrochloride, hydrobromide, sulfate, phosphate, etc.) and an organic acid salt (e.g., formate, acetate, trifuoroacetate, maleate, tartrate, methanesulfonate, benzenesulfonate, toluenesulfonate, etc.); and metal salts such as an alkali metal salt (e.g., sodium salt, potassium salt, etc.,) and an alkaline earth metal salt (e.g., calcium salt, magnesium salt, etc.).

The object compound (I) may include at least one stereoisomer such as an optical isomer (or optical isomers) and a geometric isomer (or geometric isomers) due to an asymmetric carbon atom or a double bond (or double bonds). It should be appreciated that the isomers and mixtures thereof are included in the scope of the invention.

The object compound in this invention can be prepared by the process disclosed in U.S. Pat. No. 5,521,170 and EP-A-832061.

Treatment according to this invention includes all controls, including prevention care, cure, relief or decrement of symptom, suppression of progress, etc.

The benzamide derivative of this invention is used as an active ingredient of an ocular tension depressor for use in human beings and animals. Generally, the ocular tension depressor may be administered systematically or locally by way of oral administration, intravenous administration (including drip), subcutaneous administration, rectal administration, vaginal administration, local ocular administration (including ophthalmic solutions and ophthalmic ointments), etc. Taking into account an affect to the constitution of the patient and efficacy of administration, administration in the form of local ocular preparations is particularly preferable.

For instance, ophthalmic solutions, ocular ointments, powders, granules, tablets, capsules, suppositories, suppositories for vagina, injections, and ointments are some of the forms of preparations, and preferably, the form of ophthalmic solutions and ocular ointments is suitable. These preparations can be manufactured according to known art.

According to this invention, the effective amount of the benzamide derivative is an amount necessary for carrying out a desired treatment, and varies according to the kind (human being or animal), age, and body weight of the subject to be treated, degree of symptoms of the disease to be treated, therapeutic effect demanded for the treatment, administration form, treatment duration, etc. For instance, in the case of intravenous administration, it is recommended to administer 0.1 to 1000 mg of the benzamide derivative per adult human being (more preferably, 1 to 600 mg per adult human being) a day. In the case where ophthalmic solutions containing the benzamide derivative are used as local ocular administration, it is recommended to use the solutions, in which the preparation of the benzamide derivative is dissolved at an active ingredient concentration of 0.001 to 10.0 w/v %, (preferably 0.01 to 5.0 w/v %) several times (preferably 1 to 6 times) a day each for an eye with several eye drops (preferably 1 to 4 eye drops) at a time. In the case where ocular ointment is used as local ocular administration, it is recommended to apply the ointment of the preparation containing the benzamide derivative at an active ingredient concentration of about 0.001 to 10.0 w/v % (preferably 0.01 to 5.0 w/v %) several times (preferably 1 to 6 times) a day. Any case of the above administrations brings about a satisfactory effect for the subject to be treated.

The ocular tension depressor according to this invention may contain, as an active ingredient, the benzamide derivative alone or the benzamide derivative in combination with at least one of the other pharmaceutically active ingredients. Such other pharaceutically active ingredients may include parasympathomimetic drugs (pilocarpine, carbachol, etc.), anticholinesterase agents (physostigmine salicylate, distigmine bromide, ecothiopate iodide, etc.), sympathomimetic drugs (epinephrine, dipivalylepinephrine, clonidine, p-aminoclonidine, brimonidine, etc.), β-adrenergic blocking agents (betaxolol, levobunolol, timolol, carteolol, etc), prostaglandin derivatives (isopropyl unoprostone, latanoprost), tropicamide or the like. In the preparation that contains two or more active ingredients, the amount of each ingredient may be determined appropriately according to the therapeutic effect and safety of each ingredient.

The ocular tension depressor according to this invention may contain a physiologically acceptable additive(s) as well as the aforementioned active ingredients. Such additives may include excipients, diluents, extending agents, solvents, lubricants, axuliary agents, binding agents, disintegrators, coating agents, capsules, bases for ointment, bases for suppository, aerosols, emulsifiers, dispersing agents, suspending agents, thickeners, isotonic agents, buffers, indolent agents, preservatives, antioxidants, flavoring agents, aromatic agents, coloring agents, functional agents (for example, cyclodextrin, bio-decomposable high-molecular component, etc.), stabilizers, pH regulators, and chelating agents. The kind of these additives may be determined appropriately among those of general use according to pharmaceutical practice and the amount of each additive may be determined appropriately within a therapeutically effective range.

Since the ocular tension depressor according to this invention exhibits surprisingly superb effect in depressing ocular tension, the depressor can be used to treat glaucoma, high ocular tension disease, and glaucoma with normal ocular tension.

This invention is explained in concrete in the Example described below, and the invention is not limited at all by the Example.

### EXPERIMENTAL EXAMPLE

In this Example, the following compounds A and B were used as a test compound.

[Compound A]

4-[2-(3-aminopropyl-1-yl)oxy]benzoylamino-3-methoxy-N-methyl-N-{4-methyl-2-[5-(4-methylpiperazin-1-yl)carbonylpent-1-y loxy]phenyl}benzamide

[Compound B]

5-{4-[2-(4-methylphenyl)benzoylamino]benzoyl}-1-[(4-met hyl-1-piperazinyl)carbonylmethyl]-1,3,4,5-tetrahydro-1,5-benzodi azepin-2(2H)-one

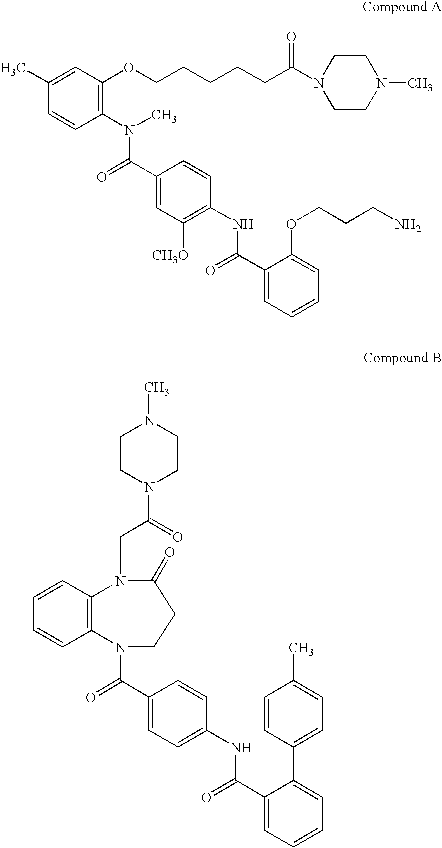

The experiment was carried out according to the following manner.

Rabbits (New Zealand albino, body weight: 2.0 to 2.5 kg) having ocular tension of 15 mmHg before treated were used in this experiment. Experimental group of the rabbits were treated with an eye drop of the preparation of a 0.5% concentration of the compounds A and B with a dosage of 50 μL at a time. Control group of the rabbits were treated with an eye drop of a medium (ophthalmic solutions containing 0.40% of NaH2PO₄, 0.47% of NaHPO₄, 0.47% of NaCl, and 1.0% of polysorbate 80 with pH 6.8) with the same dosage as in the experimental group at a time. The ocular tension of the experimental group and the control group was measured with a Tonopen™ XL tonometer before treatment, 2.5 hours, and 8 hours after treatment.

Next, a ratio of ocular tension change relative to ocular tension measured before treatment (Δ IOP %) was calculated, and an area (AUC₀₋₈ₕᵣ Δ IOP %) defined by the area under the curve obtained by plotting out Δ IOP % with respect to the horizontal axis representing lapse of time was calculated in accordance with a method of trapezoidal rule. The calculation results are shown in Table 1.

TABLE 1

| Administered | No. of Treated | AUC_{0-8 hr} Δ IOP % |
|---|---|---|
| substance | Rabbits | mean ± S.D. |
| Compound A | 4 | −147.0 ± 89.0* |
| Compound B | 4 | −145.8 ± 87.7* |
| Medium for control | 5 | −27.4 ± 49.9 |
| group | | |

*p < 0.05 (Independent samples t-test) when experimental group was compared with control group.

As is obvious from Table 1, it was verified that ocular tension of the experimental group which were administered with the object compounds A and B of this invention was significantly lowered compared with the control group.

### INDUSTRIAL APPLICABILITY

The ocular tension depressor of this invention containing the benzamide derivative represented by the formula (I) as an active ingredient is effective in depressing ocular tension. Therefore, the ocular tension depressor according to this invention is suggested to be useful for treatment of high ocular tension disease and glaucoma.

1. An ocular tension depressor containing, as an active ingredient, a compound represented by the general formula (I): 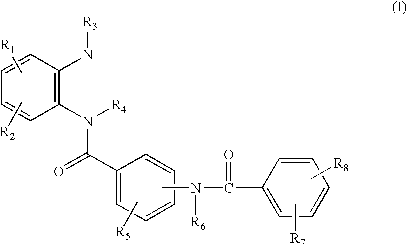
wherein
R₁ is hydrogen or lower alkyl,
R₂ is hydrogen, lower alkyl, halo(lower)alkyl, halogen or lower alkoxy,
R₃ is lower alkyl which may be substituted with acyl or acylamino,
A is O,
R₄ is hydrogen; lower alkyl which may be substituted with hydroxy, aryl or acyl; or cyclo(lower)alkyl, or
A is 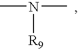
and R₉ and R₄ may be linked together to form lower alkylene which may be substituted with oxo,
R₅ is hydrogen, halogen, nitro, hydroxy, protected hydroxy, lower alkyl, or lower alkoxy which may be substituted with lower alkylamino,
R₆ is hydrogen, lower alkyl or acyl,
R₇ is hydrogen, halogen, hydroxy or lower alkoxy,
R₈ is hydroxy, aryl, acyl, amino, lower alkoxy which may be substituted with lower alkylamino or acylamino; or aryl which may be substituted with at least one substituent selected from the group consisting of lower alkyl, lower alkoxy, halogen, halo(lower)alkyl, hydroxy, amino(lower)alkyl, azido(lower)alkyl, lower alkylamino(lower)alkyl, acylamino(lower)alkyl, hydroxy(lower)alkyl, cyano and acyl, or a pharmacologically acceptable salt thereof.

2. The ocular tension depressor of claim 1, wherein
R₂ is hydrogen or lower alkyl,
R₃ is lower alkyl which is substituted with acyl,
A is O,
R₄ is lower alkyl, or
A is 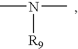
and R₉ and R₄ may be linked together to form lower alkylene which is substituted with oxo,
R₅ is hydrogen or lower alkoxy,
R₆ and R₇ are independently hydrogen,
R₈ is lower alkoxy which is substituted with amino; or phenyl which is substituted with lower alkyl.

3. The ocular tension depressor of claim 2, wherein
R₃ is lower alkyl which is substituted with N-lower alkylpiperazinylcarbonyl.

4. The ocular tension depressor of claim 3, wherein said compound is 4-[2-(3-aminopropyl-1-yl)oxy]benzoylamino-3-methoxy-N-methyl-N-{4-methyl-2-[5-(4-methylpiperazin-1-yl)carbonylpent-1-yloxy]phe nyl}benzamide.

5. The ocular tension depressor of claim 3, wherein said compound is 5-{4-[2-(4-methylphenyl)benzoylamino]benzoyl}-1-[(4-methyl-1-pip erazinyl)carbonylmethyl]-1,3,4,5-tetrahydro-1,5-benzodiazepin-2(2H)-one.

6. The ocular tension depressor of any one of claims 1 to 5 that is used to treat high ocular tension disease and/or glaucoma.

7. The ocular tension depressor of any one of claims 1 to 6 that is used in the form of preparation for ocular local administration.

8. The ocular tension depressor of claim 7 that is used in the form of ophthalmic solution.

9. A method for treating high ocular tension disease and/or glaucoma which comprises administering an effective amount of a compound represented by the general formula (I): 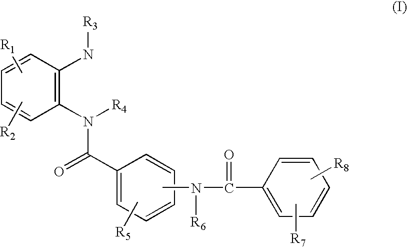
wherein
R₁ is hydrogen or lower alkyl,
R₂ is hydrogen, lower alkyl, halo(lower)alkyl, halogen or lower alkoxy,
R₃ is lower alkyl which may be substituted with acyl or acylamino,
A is O,
R₄ is hydrogen; lower alkyl which may be substituted with hydroxy, aryl or acyl; or cyclo(lower)alkyl, or
A is 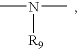
and R₉ and R₄ may be linked together to form lower alkylene which may be substituted with oxo,
R₅ is hydrogen, halogen, nitro, hydroxy, protected hydroxy, lower alkyl, or lower alkoxy which may be substituted with lower alkylamino,
R₆ is hydrogen, lower alkyl or acyl,
R₇ is hydrogen, halogen, hydroxy or lower alkoxy,
R₈ is hydroxy, aryl, acyl, amino, lower alkoxy which may be substituted with lower alkylamino or acylamino; or aryl which may be substituted with at least one substituent selected from the group consisting of lower alkyl, lower alkoxy, halogen, halo(lower)alkyl, hydroxy, amino(lower)alkyl, azido(lower)alkyl, lower alkylamino(lower)alkyl, acylamino(lower)alkyl, hydroxy(lower)alkyl, cyano and acyl, or a pharmaceutically acceptable salt thereof.

10. The method of claim 9, wherein
R₂ is hydrogen or lower alkyl,
R₃ is lower alkyl which may be substituted with acyl,
A is O,
R₄ is lower alkyl, or
A is 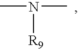
and R₉ and R₄ may be linked together to form lower alkylene which is substituted with oxo,
R₅ is hydrogen or lower alkoxy,
R₆ and R₇ are independently hydrogen,
R₈ is lower alkoxy which is substituted with amino; or phenyl which is substituted with lower alkyl.

11. The method of claim 10, wherein R₃ is lower alkyl which is substituted with N-lower alkylpiperazinylcarbonyl.

12. The method of claim 11, wherein said compound is 4-[2-(3-aminopropyl-1-yl)oxy]benzoylamino-3-methoxy-N-methyl-N-{4-methyl-2-[5-(4-methylpiperazin-1-yl)carbonylpent-1-yloxy]phe nyl}benzamide.

13. The method of claim 11, wherein said compound is 5-{4-[2-(4-methylphenyl)benzoylamino]benzoyl}-1-[(4-methyl-1-pip erazinyl)carbonylmethyl]-1,3,4,5-tetrahydro-1,5-benzodiazepin-2(2H)-one.

14. Use of a compound to treat high ocular tension disease and/or glaucoma represented by the general formula (I): 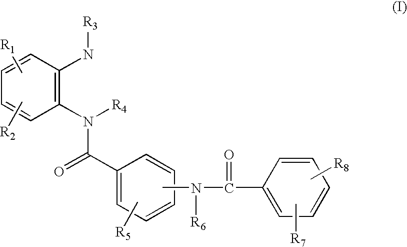
wherein
R₁ is hydrogen or lower alkyl,
R₂ is hydrogen, lower alkyl, halo(lower)alkyl, halogen or lower alkoxy,
R₃ is lower alkyl which may be substituted with acyl or acylamino,
A is O,
R₄ is hydrogen; lower alkyl which may be substituted with hydroxy, aryl or acyl; or cyclo(lower)alkyl, or
A is 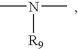
and R₉ and R₄ may be linked together to form lower alkylene which may be substituted with oxo,
R₅ is hydrogen, halogen, nitro, hydroxy, protected hydroxy, lower alkyl, or lower alkoxy which may be substituted with lower alkylamino,
R₆ is hydrogen, lower alkyl or acyl,
R₇ is hydrogen, halogen, hydroxy or lower alkoxy,
R₈ is hydroxy, aryl, acyl, amino, lower alkoxy which may be substituted with lower alkylamino or acylamino; or aryl which may be substituted with at least one substituent selected from the group consisting of lower alkyl, lower alkoxy, halogen, halo(lower)alkyl, hydroxy, amino(lower)alkyl, azido(lower)alkyl, lower alkylamino(lower)alkyl, acylamino(lower)alkyl, hydroxy(lower)alkyl, cyano and acyl, or a pharmaceutically acceptable salt thereof.

15. The use of the compound of claim 14, wherein
R₂ is hydrogen or lower alkyl,
R₃ is lower alkyl which may be substituted with acyl,
A is O,
R₄ is lower alkyl, or
A is 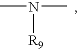
and R₉ and R₄ may be linked together to form lower alkylene which is substituted with oxo,
R₅ is hydrogen or lower alkoxy,
R₆ and R₇ are independently hydrogen,
R₈ is lower alkoxy which is substituted with amino; or phenyl which is substituted with lower alkyl.

16. The use of the compound of claim 15, wherein R₃ is lower alkyl which is substituted with N-lower alkylpiperazinylcarbonyl.

17. The use of the compound of claim 16, wherein said compound is 4-[2-(3-aminopropyl-1-yl)oxy]benzoylamino-3-methoxy-N-methyl-N-{4-methyl-2-[5-(4-methylpiperazin-1-yl)carbonylpent-1-yloxy]phe nyl}benzamide.

18. The use of the compound of claim 16, wherein said compound is 5-{4-[2-(4-methylphenyl)benzoylamino]benzoyl}-1-[(4-methyl-1-pip erazinyl)carbonylmethyl]-1,3,4,5-tetrahydro-1,5-benzodiazepin-2(2H)-one.
